(19) United States
(12) Patent Application Publication
(10) Pub. No.: US 2006/0141028 A1
(43) Pub. Date: Jun. 29, 2006

(54) Cyclosporin formulations

(76) Inventors: Moshe Flashner-Barak, Petach Tikva (IL); E. Itzhak Lerner, Petach Tikva (IL); Vered Rosenberger, Moddiin (IL); Naomi Moldavski, HaNegev (IL)

Correspondence Address: KENYON & KENYON LLP, ONE BROADWAY, NEW YORK, NY, 10004, US

(21) Appl. No.: 11/202,603

(22) Filed: Aug. 12, 2005

Related U.S. Application Data
(60) Provisional application No. 60/601,325, filed on Aug. 13, 2004.

(51) Int. Cl. A61K 38/13; A61K 31/366; A61K 31/225; A61K 31/045; A61K 9/20

(52) U.S. Cl. 424/464; 514/11; 514/460; 514/548; 514/729

(57) ABSTRACT

The present invention relates to compositions comprising solutions of drugs in menthol, especially drugs that are poorly soluble in water, and to methods for making such compositions.

### CROSS-REFERENCE TO RELATED APPLICATIONS

This application claims the benefit of provisional application Ser. No. 60/601,325, filed Aug. 13, 2004, the contents of which is incorporated herein by reference.

### FIELD OF THE INVENTION

The present invention encompasses compositions comprising solutions or solid solutions of poorly bioavailable drugs in menthol, and to methods for making such compositions.

### BACKGROUND OF THE INVENTION

Several clinically important drugs have limited oral bioavailability and high interpatient variability, resulting in difficulty in obtaining optimum treatment regimens for their use. Reasons for such limited oral bioavailability may include poor solubility in water or biological fluids, poor membrane permeability, efficient MDR (multiple drug resistance) pumps, and/or destructive metabolism in the intestine or the liver. Such metabolic destruction may be by the family of cytochrome P450 enzymes that oxidatively destroy many drugs (e.g. CYP3A4) or by glucuronidation enzymes that help the body eliminate the glucuronide derivatives of the drug in the urine or by excretion in the bile to the feces. High interpatient variability is often associated with the genetic variability of metabolic pathways in humans as well as the genetic variation in the expression of the P-glycoprotein MDR pumps.

Drugs with limited oral bioavailability include cyclosporines. Cyclosporines are a very important family of drugs which are used for the avoidance of organ rejection after organ transplant. Cyclosporines, however, suffer from erratic absorption caused by most of the factors mentioned above. See, A. Lindholm, “Factors Influencing the Pharmacokinetics of Cyclosporine in Man,” Therapeutic Drug Monitoring, 13 (6), 465-477 (1991). Cyclosporines are insoluble in water, are expelled from cells of the intestine by P-glycoprotein efflux pumps, and are heavily metabolized both in the intestine and in the liver by cytochrome P-450 enzymes. Ducharme, et al., “Disposition of Intravenous and Oral-Cyclosporine after Administration with Grapefruit Juice,” Clinical Pharmacology and Therapeutics, 57(5), 485-491 (1995); and Wu, et al., “Differentiation of Absorption and First-Pass Gut and Hepatic Metabolism in Humans: studies with Cyclosporine,” Clinical Pharmacology and Therapeutics, 58(5), 449-497 (1995). Since the therapeutic window for cyclosporines is not very wide and the toxic effects of overdose are pronounced dosing with this drug type has traditionally been difficult. See e.g., PHYSICIAN'S DESK REFERENCE, pp. 2310-2313 (57th Ed. 2003).

Cyclosporines were originally formulated in oil-based formulations so as to dissolve the drug. Oil and water do not mix very well, thus adding to the variability of the bioavailability of the product. The use of cyclosporines in microemulsion formulations has somewhat improved this situation, however, the efflux pump and oxidative metabolism issues remain essentially as problematical as before. To address the poor bioavailability of cyclosporines and other drugs, Benet and co-workers described dosing the drug either after, with, or mixed with essential oils or essential oil components such as menthol and carvone among others. See, Benet, et al., U.S. Pat. Nos. 5,665,386; 5,716,928; 6,121,234; 6,004,927; and 6,028,054. Benet showed, using in vitro tests, an inhibition of metabolism, of cyclosporine and other drugs and a concomitant improvement in bioavailability. The insolubility of the drug in water and the incompatibility of oil based formulations with the aqueous environment of the human gut were still present.

The pharmacokinetics of cyclosporine have been studied using the classical oil-based formulation and the improved microemulsion formulation along with metabolic inhibitors such as ketoconazole. Aklaghi, et al., “Pharmacokinetics of Cyclosporine in Heart Transplant Recipients Receiving Metabolic Inhibitors,” The Journal of Heart and Lung Transplantation, 20 (4), 431-438 (2001). Ketoconazole inhibits CYP3 metabolism as well as P-glycoprotein efflux pumps. The microemulsion formula gave improved bioavailability and somewhat of an improvement in variability when tested without ketoconazole. Treatment with ketoconazole greatly improved the bioavailability of the cyclosporine but not the variability. When pretreating with ketoconazole, the micro emulsion formulation was no better than the oil based formulation. While concurrent treatment with ketoconazole is practiced in certain medical centers to improve cyclosporine bioavailability, in general, the medical community is against giving potent drugs with serious toxic side effects as an adjuvant for another drug when there is no medical need for its administration. Ketoconazole is a potent anti-fungal which is known to exhibit side effects. The need for a safe alternative that will both raise the bioavailability of cyclosporines and lower the inter-patient variability is still present.

The present invention overcomes many of the existing limitations within the prior art by providing novel formulations.

### SUMMARY OF THE INVENTION

One embodiment of the invention encompasses compositions for reducing the variability of the bioavailability of a drug comprising at least one poorly bioavailable drug dissolved in an effective amount of menthol. The poorly bioavailable drug may be at least one drug with low aqueous solubility, a drug metabolized by cytochrome P450, a drug expelled from cells by the P-glycoprotein pump, or a drug metabolized via glucuronidation. A drug with low aqueous solubility is a drug having a water solubility of less than about 20 mg/per milliliter of water. In one embodiment, the amount of menthol may be from about 20% to about 99% by weight, preferably, preferably the menthol may be present in an amount of about 60% to about 95% by weight of the composition.

One embodiment of the invention encompasses a composition for reducing the variability of the bioavailability of a drug comprising cyclosporine dissolved in an effective amount of menthol and at least one surface active agent. In the composition, cyclosporine is present in an amount of about 5% to about 20% by weight and menthol is present in an amount of about 30% to about 80% by weight. In the composition, the surface active agent is present in an amount of about 20% to about 60% by weight. The surface active agent may comprise a first surface active agent and a second surface active agent. If two surface active agents are present, then the first surface active agent is present in an amount of about 25% to about 35% by weight and the second surface active agent is present in an amount of about 10% to about 20% by weight.

Yet another embodiment of the invention encompasses methods for reducing the variability of the bioavailability of a drug comprising dissolving at least one poorly bioavailable drug in an effective amount of menthol. Optionally, the method may comprise at least one surface active agent. The method may further comprise administering the composition to a mammal. In one embodiment of the method, the amount of menthol may be sufficient to decrease the variability in the drug's bioavailability by about 10% or more of the relative standard deviation (CV %) of the area under the blood or plasma concentration versus time curve (AUC) when compared to the AUC of a non-menthol containing formulation of the drug.

Another embodiment of the invention encompasses methods for increasing the extent of time that a drug provides a therapeutically significant concentration in blood or plasma comprising dissolving at least one poorly bioavailable drug in an effective amount of menthol. In one embodiment, the amount of menthol may be sufficient to extend the time that the drug provides a therapeutically significant concentration in blood or plasma by one hour or more.

Yet another embodiment of the invention encompasses a composition for improving the bioavailability of cyclosporine comprising about 10% by weight cyclosporine dissolved in about 40% menthol, about 34% Tween 80, and about 17% ducosate sodium, wherein the weights are in percents of the composition. The composition has an AUC_{I} of about 248 ng*hr/ml and/or a Cₘₐₓ of about 69.9 ng/ml.

Another embodiment of the invention encompasses a composition for improving the bioavailability of a drug comprising simvastatin dissolved in an effective amount of menthol. The simvastatin is present in an amount of about 10% by weight.

One embodiment of the invention encompasses a composition for improving the bioavailability of a drug comprising simvastatin dissolved in an effective amount of menthol, wherein the composition has an average AUCₜ of about 181% as compared to a non-menthol containing formulation. The simvastatin composition has an average AUC_{I} of about 200% and/or an average Cₘₐₓ of about 143% as compared to a non-menthol containing formulation.

Yet another embodiment of the invention encompasses a composition for improving the bioavailability of a drug comprising simvastatin dissolved in an effective amount of menthol, wherein the composition has an average AUC_{I} of about 127% as compared to the sequential administration of simvastatin and menthol. The simvastatin composition has an average Cₘₐₓ of about 138% as compared to the sequential administration of simvastatin and menthol.

Yet another embodiment of the invention encompasses a composition for improving the bioavailability of an active metabolite of a drug comprising simvastatin dissolved in an effective amount of menthol, wherein the composition has an average AUCₜ for simvastatin hydroxyacid of about 143% as compared to a non-menthol containing formulation. The composition has an average AUCₜ for the simvastatin hydroxyacid of about 124% as compared to the sequential administration of simvastatin and menthol. The composition has an average Cₘₐₓ for the simvastatin hydroxyacid of about 145% as compared to a non-menthol containing formulation. The composition has an average Cₘₐₓ for the simvastatin hydroxyacid of about 129% as compared to the sequential administration of simvastatin and menthol.

Another embodiment of the invention encompasses a composition for reducing the variability of the bioavailability of an active metabolite of a drug comprising simvastatin dissolved in an effective amount of menthol, where the composition has a % CV for AUCₜ for simvastatin hydroxyacid which is at least about 30% lower when compared to a non-menthol containing formulation.

Another embodiment of the invention encompasses a composition for improving the bioavailability of a drug comprising raloxifene HCl and an effective amount of menthol, wherein the raloxifene HCl and the menthol are administered concomitantly. The raloxifene may be present in an amount of about 25% by weight of the composition.

One embodiment of the invention encompasses a composition for improving the bioavailability of a drug comprising raloxifene HCl and an effective amount of menthol, wherein the raloxifene HCl and the menthol are administered concomitantly and the composition has an average AUC of about 108% as compared to a non-menthol containing formulation. The raloxifene HCl composition has an average Cₘₐₓ of about 136% as compared to a non-menthol containing formulation.

### DETAILED DESCRIPTION OF THE INVENTION

The invention comprises formulations of drugs with low bioavailability and menthol. As used herein, the term “poor bioavailability” or “poorly bioavailable” refers to a drug that has an oral bioavailability in its active form, whether it be the drug as dosed or an active metabolite thereof, of less than 30%.

Not to be limited by theory, it is believed that the compositions of the invention operate, in part, by providing a composition where the poorly bioavailable drugs are combined with compounds that aid solubility and/or compounds that compete with the poorly bioavailable drug in the biodegradable pathway which degrades the poorly bioavailable drugs. The delivery of the poorly bioavailable drugs is improved by using materials that are generally recognized as safe and without the use of potent drugs to establish an efficient competition within the biodegradable pathway. Thus, the non-active compound would be metabolized prior to the active drug. Menthol containing compositions and methods of using menthol in drug compositions are further described in U.S. publication No. 2004/198646, published on Oct. 7, 2004, the contents of which are incorporated herein by reference.

In particular, in the studies we found that formulating poorly bioavailable drugs as a solution or a solid solution in menthol improved delivery as compared to dosing the drug alone, dosing the drug after a menthol dose or a menthol containing dose (e.g. peppermint oil), or dosing the drug along with a dose of menthol. The compositions of the invention allow for the use of lower doses of drugs that provide the same systemic concentrations of drugs as the currently supplied doses that undergo extensive presystemic metabolism and degradation. Also, the compositions of the invention reduce interpatient variability caused by the inherently differing metabolic profiles between subjects. In particular, cyclosporine exhibits poor water solubility and is currently dosed as an oil solution containing surface active agents to aid solubility in the form of a microemulsion. One embodiment of the invention dissolves cyclosporine in menthol and adds at least one surface active agent to reduce the variability of cyclosporine absorption.

Menthol, chemically known as (1α,2β,5α)-5-methyl-2-(1-methylethyl)-cyclohexanol, is partially soluble in water. Because menthol has a low melting point, i.e., about 41° C. to 43° C., compositions of menthol and drugs dissolved within the menthol have melting points close to body temperature. This property allows menthol to act as an efficient solvent for many drugs. We have found that menthol is a superior solvent for poorly water soluble drugs as compared to oil based drug formulations, because, in part, the drugs are more available to the aqueous medium of the gastro-intestinal tract as compared to oil based formulations. Although, menthol has been known to act as a skin absorption enhancer, it is believed that menthol may also improve gastro-intestinal drug absorption as well.

The invention advantageously uses menthol in association with poorly bioavailable drugs to deter drug biodegradation in a kinetically competitive environment. In other words, menthol may be used to inhibit biological degradation pathways which metabolize the active drug and/or kinetically compete with the drug at the biologically active degradation site. For example, menthol inhibits CYP3A4 metabolism and the P-glycoprotein pump; thus, menthol in association and in intimate contact with the poorly bioavailable drug greatly enhances the bioavailability of the drug as the drug does not undergo degradation. Also, menthol, which has been shown to be metabolized to a glucuronide derivative, can serve as a sacrificial molecule wherein menthol is degraded prior to the drug, thus delaying drug degradation and extending drug bioavailability. In other words, menthol is potentially capable of competing with a drug as a decoy for glucuronidation, thereby leaving less of the drug metabolized and yielding an overall increase in the drug bioavailability.

The present invention encompasses pharmaceutical compositions for improving the bioavailability of a drug comprising at least one drug dissolved in an effective amount of menthol. In particular, the invention encompasses pharmaceutical compositions for improving the bioavailability of a drug comprising at least one poorly bioavailable drug dissolved in an effective amount of menthol. Optionally, the compositions may additionally comprise at least one surface active ingredient. As used herein, the term “improving bioavailability” refers to the increase in blood or plasma concentration of a drug dosed with menthol as compared to the blood or plasma concentration of the drug when dosed without menthol. In other words, drug bioavailability is proportional to, and is typically measured by, the total area under the curve (AUC) of the concentration of the drug found in blood or plasma versus time when measured in a pharmacokinetic trial in a human or an animal. The AUC may be expressed as AUCₜ, i.e. the area under the curve to the last measured time point, or AUC_{I}, i.e. the area under the curve extrapolated to infinite time. The improvement in bioavailability is measured by the percent increase in the average AUC of the subjects in the trial when dosing the drug dissolved in menthol as compared to the average AUC of the same subjects obtained by standard dosing of the drug. Alternately, the AUC ratio of the test formulation (AUC_{f}) to the AUC of the reference formulation (AUCᵣ) may be calculated on a per subject basis and then averaged. A percent of the average ratio (AUC_{f}/AUCᵣ) above 100% is then the improvement in bioavailability. Typically, the improvement in the average AUC when dosing the drug dissolved in menthol as compared to the average AUC obtained by standard dosing of the drug is about 5%, and preferably, the improvement is about 10% or more in the bioavailability, which is considered significant.

The present invention further provides a pharmaceutical composition directed to improving the extent of time that a drug provides a therapeutically significant concentration in blood or plasma and/or reducing the drug bioavailability variability, wherein the drug is dissolved in menthol. As used herein, the term “improving the extent of time” refers to the increase in length of time that a drug provides a therapeutically effective concentration in blood or plasma. Preferably, the time a drug provides a therapeutically effective concentration in blood or plasma is extended by about one hour or more. As used herein, the term “drug bioavailability variability” is defined as the relative standard deviation, expressed as CV %, of the drug's AUC over the subjects tested. A highly variable drug is one with a CV % greater than 50%. An improvement of the CV % by 10 percent or more is considered significant. The present invention is particularly directed to a pharmaceutical composition comprising a solid or solid solution of a drug dissolved in an effective amount of menthol. The solid solution may include a compound or polymer that forms a dispersion with the drug.

The poor bioavailability of the drug may be due to several factors. Such factors include, but are not limited to, low aqueous solubility, metabolism by cytochrome P450, expulsion from cells by the P-glycoprotein pump, or metabolism via glucuronidation. Thus, the present invention encompasses compositions for increasing the bioavailability of drugs with low aqueous solubility, drugs metabolized by cytochrome P450, drugs expelled from cells by the P-glycoprotein pump, and/or drugs metabolized via glucuronidation. As used herein, the term “low aqueous solubility” refers to a drug that is considered to be poorly water-soluble, i.e., the drug has a water solubility of less than about 20 mg per milliliter of water.

Any pharmacologically active substance or drug can be used in the practice of the present invention. Preferred drugs, however, include drugs having poor bioavailability. Examples of drugs having poor bioavailability include, but are not limited to, cyclosporine, statins, paclitaxel, fenofibrate, itraconazole, bromocriptine, carbamazepine, diazepam, paclitaxel, etoposide, camptothecin, danazole, progesterone, nitrofurantoin, estradiol, estrone, oxfendazole, proquazone, ketoprofen, nifedipine, verapamil, or glyburide. Statins include, but are not limited to, atorvastatin, cerivastatin, fluvastatin, lovastatin, mevastatin, pravastatin, or simvastatin. Preferably, the drugs having poor availability include at least one of cyclosporine, statins, or paclitxel. More preferred drugs include simvastatin or cyclosporine. Other examples of drugs having poor bioavailability will be readily apparent to one of ordinary skill in the art.

The amount of drug in the composition of the invention should be sufficient to be therapeutically effective for the condition administered. One of ordinary skill in the art can easily determine with little or no experimentation the effective amount of drug. Typically, the drug is present in an amount of about 5% to about 40% by weight of the composition, preferably, the drug is present in an amount of about 10%.

The amount of menthol in the composition of the invention should be sufficient to improve the reducing the bioavailability variability of the poorly bioavailable drug. Typically, the amount of improvement should be at least about 5% of the average AUC as compared to the average AUC of a non-menthol containing formulation and preferably, the improvement is about 10%. More preferably, the amount of improvement should be at least about 15% of the average AUC as compared to the average AUC of a non-menthol containing formulation. One of ordinary skill in the art can easily determine with little or no experimentation the effective amount of menthol. Typically, menthol is present in the composition in a amount of about 20% to about 99% by weight of the composition, and preferably, menthol is present in an amount of about 60% to about 95%. More preferably, menthol is present in the composition in an amount of about 80% to about 90% by weight.

A preferred embodiment of the invention encompasses solutions comprising menthol, cyclosporine, and at least one surface active agent. Cyclosporine may be present in an amount of about 5% to about 20% by weight, preferably about 5% to about 15%, and more preferably, cyclosporine is present in an amount of about 9% to about 11% by weight. Menthol may be present in an amount of about 30% to about 80% by weight, preferably about 30% to about 50%, and more preferably, menthol may be present in an amount of about 38% to about 42% by weight. The surface active agent may be present in an amount of about 20% to about 60% by weight. Preferably, two surface active agents are present the first in an amount of about 25% to about 35% by weight and the second in an amount of about 10% to about 20% by weight. More preferably, when two surface active agents are present, the first is present in an amount of about 30% to about 35% by weight and the second is present in an amount of about 15% to about 18% by weight. One of ordinary skill in the art readily recognizes suitable surface active ingredients. For example, surface active agents include, but are not limited to, Tween 80 or ducosate sodium.

Particular embodiments of the invention include compositions comprising about 5% to about 20% cyclosporine, about 30% to about 80% menthol, and about 20% to about 60% of at least one surface active agent by weight. Another embodiment comprises about 5% to about 15% cyclosporine by weight, about 30% to about 50% menthol, about 25% to about 35% of a first surface active agent, such as Tween 80, and about 10% to about 20% of a second surface active agent, such as ducosate sodium. A more preferred embodiment comprises about 9% to about 11% cyclosporine by weight, about 38% to about 42% menthol, about 30% to 35% of a first surface active agent, such as Tween 80, and about 15% to about 18% of a second surface active agent, such as ducosate sodium.

The compositions of the invention may also encompass other excipients commonly used in drug manufacture including, but not limited to, binders, fillers, disintegrants, lubricants, colorants, carriers, and diluents.

Another embodiment of the invention encompasses methods of improving the bioavailability of a drug comprising dissolving the drug in an effective amount of menthol. In particular, the invention encompasses methods for improving the bioavailability of a drug comprising dissolving at least one drug with low aqueous solubility, drug capable of being metabolized by cytochrome P450, a drug capable of being expelled from cells by the P-glycoprotein pump, or a drug capable of being metabolized via glucuronidation in an effective amount of menthol and optionally in the presence of a surface active ingredient. Typically, the amount of improvement should be at least about 5% of the average AUC as compared to the average AUC of a non-menthol containing formulation and preferably about 15%, as explained above.

The invention encompasses methods for reducing the variability of the bioavailability of a drug comprising dissolving at least one drug with low aqueous solubility, a drug capable of being metabolized by cytochrome P450, a drug capable of being expelled from cells by the P-glycoprotein pump, or a drug capable of being metabolized via glucuronidation in an effective amount of menthol and optionally in the presence of a surface active ingredient. As described above, drug variability is defined as the relative standard deviation, expressed as CV %, of the drug's AUC over the subjects tested. A highly variable drug is one with a CV % greater than 50%. Typically, the reduction is about 5% of the relative standard deviation (CV %) of the area under the blood or plasma concentration versus time curve (AUC) when compared to a non-menthol containing formulation average AUC, and preferably, the decrease in CV % is by about 10% or more, which is considered significant.

Another embodiment of the invention encompasses methods for delivering cyclosporine comprising administering a menthol solution of cyclosporine with at least one surface active agent. A preferred method of delivering cyclosporine comprises the administration cyclosporine, menthol, and at least one surface active agent in the compositions of the invention as described above.

Another embodiment of the invention encompasses methods for increasing the extent of time that a drug provides a therapeutically significant concentration in blood or plasma comprising dissolving at least one poorly bioavailable drug in an effective amount of menthol, optionally in the presence of at least one surface active agent. Typically, the extent of the bioavailability of a drug is increased by the administration of a composition comprising at least one drug and menthol, wherein the menthol is present in an amount sufficient to extend the time that the drug provides a therapeutically significant concentration by one hour or more.

The present invention encompasses unit dosage forms of the pharmaceutical composition comprising a unit dosage form of a drug dissolved in an effective amount of menthol. The unit dosage form may optionally contain at least one surface active agent. The compositions of the invention may be administered to a mammal. Preferably, the mammal is a human.

One embodiment encompasses the compositions of the invention be prepared into solid solution dosage forms. In particular, the compositions may be formulated into oral solid dosage forms such as capsules, tablets, or gelcaps. In particular, the pharmaceutical compositions can be made into unit dosage forms.

In one embodiment, the solid solution is formed on the surface of at least one pharmaceutical carrier particle. For example, a molten combination of drug and menthol can be applied to the surface of particles of one or more pharmaceutical carriers, and allowed to cool to form the solid solution on the surface of the pharmaceutical carrier or carriers.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### Examples

### Example 1

Cyclosporine (20 g) was heated in menthol (80 g) to 56° C. while stirring until the cyclosporine dissolved yielding a clear solution. Microcrystalline cellulose (Avicel pH 102, 100 g) was added to the clear solution which was cooled to room temperature giving a solid solution of cyclosporine in menthol on the microcrystalline cellulose. The solid was milled using a Quadro Comil milling machine, with screens of 6350, 1575 and 813 microns sequentially used to produce a powder ready for filling into capsules.

### Example 2

Simvastatin (20 g) was heated in menthol (200 g) to 60° C. while stirring at 150 rpm in a jacketed reactor. The simvastatin dissolved in the menthol to give a clear solution. The solution was cooled to room temperature to a solid solution of simvastatin in menthol. The solid solution was milled using a Quadro Comil milling machine with a 1640 micron screen. The powder (200 mg) was filled into #0 capsules. The capsules were assayed for simvastatin content by dissolving a capsule in a pH 4 phosphate buffer containing acetonitrile (1:1). The simvastatin content was assayed on a C-18 column by HPLC and found to contain 20 mg of simvastatin per capsule. The release of simvastatin was measured in 450 ml of pH=7 phosphate buffer containing 0.5% sodium lauryl sulfate (SLS) in water at 37° C. and 50 rpm in an USP apparatus II dissolution system. The release was found to be greater than 75% at 30 minutes.

### Example 3

Raloxifene HCl (60 mg, Evista, ELI LILLY®) was dosed to twelve healthy volunteers either alone or with a capsule containing 180 mg menthol in a crossover fashion with a two week washout between sessions. Blood samples were taken at 0, 0.5, 1, 2, 4, 6, 8, 10, 12, 16, 20, 24, 48, 72 and 96 hours and the content of raloxifene assayed. The average Cₘₐₓ of the raloxifene dosed with menthol was 36% higher than the reference (320 pg/ml vs. 235 pg/ml), while the average area under the curve (AUC) was 8% higher when dosing with menthol (13041 vs. 12090 pg*hr/ml). Raloxifene is a long half-life drug (t_{1/2} for the test was 26 hours and for the reference was 28 hours), while menthol has a short half-life. Without wishing to be bound by theory or mode of action, it is believed that the main effect of menthol is seen in the first hours where it can effectively compete with the drug for glucuronization. An analysis of the AUC over the first six hours shows that the test AUC is 35% higher than the reference, mirroring the Cₘₐₓ result. Without wishing to be bound by theory or mode of action, it is believed that dosing with menthol can successfully compete with the metabolism of the drug, yielding a better pharmacokinetic profile.

### Example 4

An open-label study with randomized three-way crossover comparative pharmacokinetic study was conducted with 12 healthy fasted volunteers each receiving a single dose of either: Reference-simvastatin (Simvastatin-Teva®, 20 mg) alone; Test 1-simvastatin (Simvastatin-Teva®, 20 mg)+menthol (180 mg capsule); or Test 2-simvastatin/menthol (10% simvastatin dissolved in menthol, 20 mg of simvastatin per capsule). A dose was administered to each subject on three occasions, separated by at least a 1 week wash-out period between each session. All subjects received both the tests and reference drugs in a three-way crossover design.

Each subjects was randomly assigned at the first study period to either of the Test formulations or to the Reference formulation, and was subsequently crossed over at least one week later to either of the alternative treatments. The process was repeated during the third study session, such that each subject was exposed to one of the following treatment schemes: T₁→R→T₂; T₁→T₂→R; R→T₁→T₂; R→T₂→T_{1;} T₂→R→T₁; T₂→T₁→R.

Drug concentration was determined by taking blood samples from all subjects regardless of treatment assignment at the following time points: 0 hour (pre-dosing), 0.5, 1, 1.5, 2, 3, 4, 6, 8, 10 and 12 hours post-initial dosing, for a total of 11 samples per study. Each sample was tested for simvastatin lactone and simvastatin hydroxyacid, the active metabolite, by analysis using a validated LC/MS/MS method.

The AUCₜ and AUC_{I}, Cₘₐₓ, Tₘₐₓ, and half life (t_{1/2}) were calculated for each volunteer both for simvastatin in plasma and for the active metabolite simvastatin hydroxyacid in plasma. Table 1 illustrates the average values for simvastatin in plasma and compares the values of the two test formulations to the average values obtained with the reference formulation.

TABLE 1
Simvastatin Concentration in Plasma

| | | | | Test1/ | Test2/ |
|---|---|---|---|---|---|
| | Test 1 | Test 2 | Reference | Ref. | Ref. |
| Avg. AUCₜ (ng * h/ml) | 20.8 | 26.9 | 14.9 | 1.4 | 1.81 |
| Avg. AUC_{I} ng * h/ml) | 25.9 | 33.0 | 16.3 | 1.59 | 2.02 |
| Avg. Cₘₐₓ (ng/ml) | 5.8 | 8.0 | 5.6 | 1.04 | 1.43 |
| Avg. Tₘₐₓ (hours) | 1.58 | 2.46 | 1.04 | | |
| Avg. t_{1/2} (hours) | 3.99 | 3.10 | 2.03 | | |

Table 1 demonstrates that both test formulations showed improved bioavailability over the simvastatin reference with the sample having 20 mg of simvastatin dissolved in 180 mg of menthol giving even better results than the concomitant dosing of a 20 mg simvastatin tablet along with a capsule of 180 mg of menthol. For AUCₜ the average improvement in the bioavailability of Test 1 (concomitant separate dosing) the improvement was 40% while the improvement for the drug dissolved in menthol was 81%. The corresponding values for the AUC extrapolated to infinity were 59% and 102%, respectively. Consequently, the dissolved product gave larger improvements than concomitant separate dosing.

The ratio of the AUCₜ of each test formulation to the reference formulation for each volunteer was calculated (each volunteer being his own control) and the average value of the ratio calculated. These results are illustrated in Table 2.

TABLE 2
Ratio Analysis of AUCt for Simvastatin in Plasma

| Subject | Test 1 | Test 2 | Reference | Test 1/Ref. | Test 2/Ref. |
|---|---|---|---|---|---|
| 1 | 20.78 | 21.43 | 27.75 | 0.749 | 0.772 |
| 2 | 29.54 | 39.70 | 38.96 | 0.758 | 1.02 |
| 3 | 23.53 | 17.62 | 6.76 | 3.48 | 2.61 |
| 4 | 26.89 | 75.45 | 33.12 | 0.812 | 2.28 |
| 5 | 37.32 | 15.08 | 6.68 | 5.59 | 2.26 |
| 6 | 12.7 | 10.85 | 4.31 | 2.95 | 2.52 |
| 7 | 8.18 | 6.26 | 5.42 | 1.51 | 1.16 |
| 8 | 13.48 | 26.46 | 13.85 | 0.975 | 1.91 |
| 9 | 31.96 | 66.15 | 13.53 | 2.36 | 4.89 |
| 10 | 22.29 | 23.63 | 15.29 | 1.46 | 1.55 |
| 11 | 14.66 | 9.55 | 7.75 | 1.89 | 1.23 |
| 12 | 8.79 | 10.68 | 5.17 | 1.70 | 2.07 |
| Mean | 20.8 | 26.9 | 14.9 | 2.02 | 2.02 |
| ±SD | 9.4 | 22.5 | 11.9 | 1.42 | 1.09 |
| CV % | 45.3 | 83.7 | 80.1 | 70.5 | 54 |

Table 2 illustrates the ratio analysis of the AUCₜ values. Both test formulations showed a more than 100% improvement in bioavailability compared to the reference formulation. The two test formulations gave the same larger improvement. The value for Tₘₐₓ is somewhat delayed for Test 2 compared to the reference and slightly so for Test 1. The values of t_{1/2} are slightly longer, which may indicate competition by menthol for metabolic pathways that determine the t_{1/2} such a glucuronidation and CYP3A4 pathways.

Table 3 collected the average values for simvastatin hydroxyacid, the active metabolite, in plasma and compared the values of the two test formulations to the average values obtained with the reference formulation.

TABLE 3
Simvastatin Hydroxyacid in Plasma

| | | | | Test 1/ | Test 2/ |
|---|---|---|---|---|---|
| | Test 1 | Test 2 | Reference | Ref. | Ref. |
| Avg AUCₜ (ng * h/ml) | 10.2 | 12.7 | 8.9 | 1.15 | 1.43 |
| Avg Cₘₐₓ (ng/ml) | 1.32 | 1.71 | 1.18 | 1.12 | 1.45 |
| Avg Tₘₐₓ (hours) | 5.5 | 5.4 | 5.4 | | |
| Avg t_{1/2} (hours) | 8.8 | 5.7 | 6.5 | | |

Table 3 illustrates the values for the active metabolite of simvastatin. Both test formulations showed improved bioavailability as expressed as average AUCₜ. Test 1 (concomitant separate dosing) showed a 15% improvement in the average bioavailability of the active moiety when compared to the reference drug product. Test 2 (concomitant dissolved dosing) showed a 43% improvement in the average AUCₜ and therefore in average bioavailability. Test 2 showed also a 45% improvement in the average Cₘₐₓ. Table 3 illustrates also that the composition for Test 2 has an average AUCₜ for the simvastatin hydroxyacid of about 124% as compared to the sequential administration of simvastatin and menthol. The composition for Test 2 also has an average Cₘₐₓ for the simvastatin hydroxyacid of about 129% as compared to the sequential administration of simvastatin and menthol.

The ratio of AUCₜ of each test formulation to the reference formulation for each volunteer was calculated (each volunteer being his own control) and the average value of the ratio calculated. These results are illustrated in Table 4.

TABLE 4
Ratio Analysis of AUCₜ for Simvastatin Hydroxyacid in Plasma

| Subject | Test 1 | Test 2 | Reference | Test 1/Ref. | Test 2/Ref. |
|---|---|---|---|---|---|
| 1 | 9.75 | 7.44 | 10.49 | 0.927 | 0.710 |
| 2 | 17.28 | 19.10 | 25.99 | 0.665 | 0.735 |
| 3 | 9.57 | 11.66 | 5.20 | 1.84 | 2.24 |
| 4 | 7.85 | 18.49 | 8.04 | 0.976 | 2.30 |
| 5 | 13.23 | 9.05 | 5.52 | 2.40 | 1.64 |
| 6 | 7.61 | 11.3 | 5.18 | 1.47 | 2.18 |
| 7 | 9.55 | 10.88 | 7.29 | 1.31 | 1.49 |
| 8 | 3.76 | 5.46 | 4.18 | 0.900 | 1.31 |
| 9 | 9.65 | 17.77 | 4.70 | 2.06 | 3.78 |
| 10 | 19.24 | 21.77 | 16.37 | 1.18 | 1.22 |
| 11 | 12.69 | 13.61 | 10.97 | 1.16 | 1.24 |
| 12 | 2.64 | 6.37 | 2.73 | 0.967 | 2.33 |
| Mean | 10.2 | 12.7 | 8.9 | 1.32 | 1.77 |
| ±SD | 4.9 | 5.4 | 6.6 | 0.53 | 0.85 |
| CV % | 47.5 | 42.5 | 73.9 | 39.8 | 48.1 |

Table 4 illustrates the ratio analysis for the AUCₜ values for the active moiety. Both test formulations showed a clear improvement in the average of the individual ratios of AUCₜ with Test 2 being superior to Test 1. Test 1 showed an improved ratio of 32% compared to the reference drug product while Test 2 showed a 77% improvement. The variability of the drug absorption for the active moiety is also clearly improved when dosing with menthol. The reference had a percent coefficient of variation of 74% while Test 1 showed 48% and Test 2 showed 43%, both a considerable improvement and Test 2 being superior. Table 4 illustrates also that the % CV for AUCₜ for simvastatin hydroxyacid is at least about 30% lower when compared to a non-menthol containing formulation.

Therefore, administering simvastatin with menthol can significantly improve the bioavailability of both the parent drug and its active metabolite and delivering the drug when dissolved in the menthol gives an even greater improvement in the improved bioavailability, and a lower variability of the active moiety. The approximate 80 to 100% improvement in the bioavailability of the simvastatin itself and the simvastatin hydroxyacid active moiety along with lowered variability should be able to lead to improved dosing and treatment with this important drug.

### Example 5

Samples for a pharmacokinetic trial were prepared by placing within a double walled glass reactor heated to 65° C., menthol (75 g from EP), Tween 80 (63.75 g from Uniqema), and ducosate sodium (31.5 g from USP). The mixture was stirred at 200 rpm until a melt solution was formed and thereafter, cyclosporine (18.75 g from Poli Industria Chimica SPA) was added and the melt was stirred at 200 rpm until full dissolution. Capsules size “0” were filled with the melt solution (252 mg±12 mg). After analysis, the capsules were found to have 25.2 mg±1.6% RSD of cyclosporine in a viscous liquid. This formulation was used as Test formulation #2 (treatment C) below.

The trial was conducted as an open-label, randomized, single-dose, 3-way crossover comparative bioavailability study. The study was designed to determine the intersubject variability of AUC₀₋ₜ, AUC_{inf}, and Cₘₐₓ, at each endpoint. The sample group consisted of twelve healthy, male, non-smoking adult volunteers. Each volunteer was administered one of three treatments. The first treatment, treatment A, comprised of administering a 1×25 mg soft gelatin cyclosporine capsule (Neoral®, Novartis Lot #-173H0395); the second treatment, treatment B, comprised administration of a 1×25 mg cyclosporine capsule containing cyclosporin dissolved in neat methanol, labeled Test formulation #1; and the third treatment, treatment C, comprised of administration of a 1×25 mg cyclosporine capsule, labeled Test formulation #2. Each volunteer was administered a single oral dose of 25 mg administered with 240 ml water.

Blood samples (1×5 ml) were collected in EDTA containing tubes before dosing and at 0.5, 0.75, 1, 1.25, 1.5, 1.75, 2, 2.5, 3, 4, 5, 6, 8, 10, 12, 16, 20, 24, 28, and 32 hours post dosing. In between trials, a washout period of at least 14 days was implemented. The samples were analyzed for cyclosporine in whole blood using an LC/MS/MS method validated for the range of 1 to 100 ng/ml.

Tables 5 and 6 summarized the results of the pharmacokinetic study.

TABLE 5
Summary of log-transformed pharmacokinetic results

| | | | | | |
|---|---|---|---|---|---|
| | AUC₀₋ₜ | AUC_{inf} | | | T_{1/2} |
| Parameter | (ng * hr/ml) | (ng * hr/ml) | Cₘₐₓ (ng/ml) | Tₘₐₓ (hr) | (hr) |

Treatment A (Reference)

| | | | | | |
|---|---|---|---|---|---|
| Mean | 247.7 | 263.4 | 85.2 | 1.33 | 7.27 |
| % CV | 34.9 | 33.3 | 40.8 | 32.3 | 43.8 |
| N | 12 | 12 | 12 | 12 | 12 |

Treatment B

| | | | | | |
|---|---|---|---|---|---|
| Mean | 60.3 | 79.6 | 14.9 | 3.08 | 4.89 |
| % CV | 84.0 | 55.8 | 82.4 | 37.1 | 77.0 |
| N | 12 | 11 | 12 | 12 | 11 |

Treatment C

| | | | | | |
|---|---|---|---|---|---|
| Mean | 236.1 | 248.6 | 69.9 | 1.35 | 6.87 |
| % CV | 18.0 | 19.5 | 22.6 | 23.4 | 39.9 |
| N | 10 | 9 | 10 | 10 | 9 |

Table 5 summarizes the results of the trial using log-transformed data. Treatment A, the reference product, gave a mean AUC₀₋ₜ of 247.7 (ng*hr/ml) and a mean Cₘₐₓ of 85.2 (ng/ml). The reference product was an emulsion of cyclosporine in oil and surface active agents commonly marketed at Neoral® by Novartis Inc. Treatment B, cyclosporine dissolved in menthol alone, gave lower values than the reference product for both the AUC and Cₘₐₓ. It is believed that the result may be due to cyclosporine's hydrophobicity which prevented cyclosporine from leaving the menthol phase and thus, hindered drug absorption. Treatment C, cyclosporine dissolved in menthol with the addition of surface active agents, gave an AUC₀₋ₜ of 236.1 (ng*hr/ml), which is very similar to that of the reference product, and a lower Cₘₐₓ of 69.9 (ng/ml). A comparison of Treatment C with the reference product yielded an AUC value that was 96% of that of the reference, while the Cₘₐₓ was 82% of the reference.

Drug efficacy is related to the AUC while adverse effects are usually related to drug levels above the necessary therapeutic window (both AUC and Cₘₐₓ). Based on the data, Treatment C, with a lower Cₘₐₓ but same AUC, was superior in drug delivery than the reference product. More importantly, Treatment C showed less variation in both the overall AUC and the Cₘₐₓ. The % CV for AUC₀₋ₜ for Treatment A was 34.9% while it was 18% for Treatment C. The % CV for the Cₘₐₓ value was 40.8% for Treatment A and 22.6% for Treatment C. The variability of Treatment C was about half of that of the reference treatment, a significant improvement for therapeutic use of the drug.

TABLE 6
Individual Pharmacokinetic Parameters

| | Treat. | | | | | | | | |
|---|---|---|---|---|---|---|---|---|---|
| | A (ref) | A | A | B | B | B | C | C | C |
| | AUC₀₋ₜ | Cₘₐₓ | Tₘₐₓ | AUC₀₋ₜ | Cₘₐₓ | Tₘₐₓ | AUC₀₋ₜ | Cₘₐₓ | Tₘₐₓ |
| Subject | (ng * hr/ml) | (ng/ml) | (hr) | (ng * hr/ml) | (ng/ml) | (hr) | (ng * hr/ml) | (ng/ml | (hr) |
| 1 | 224.3 | 84.1 | 1.25 | 96.2 | 22.2 | 2.00 | 215.5 | 71.6 | 1.75 |
| 2 | 486.6 | 149.0 | 1.25 | 63.1 | 15.0 | 2.50 | 301.3 | 85.0 | 1.75 |
| 3 | 206.4 | 83.0 | 1.50 | 58.4 | 27.3 | 2.00 | 274.1 | 93.3 | 1.00 |
| 4 | 203.4 | 53.3 | 2.50 | 146.4 | 39.2 | 2.00 | 236.8 | 62.3 | 1.75 |
| 5 | 203.7 | 79.9 | 1.00 | 78.8 | 16.2 | 4.00 | 216.0 | 63.8 | 1.50 |
| 6 | 375.9 | 117.0 | 1.50 | 97.3 | 15.7 | 5.00 | — | — | — |
| 7 | 246.5 | 78.0 | 1.50 | 27.2 | 6.3 | 2.00 | 221.9 | 80.0 | 1.25 |
| 8 | 133.0 | 37.6 | 1.25 | 36.5 | 11.0 | 2.50 | — | — | — |
| 9 | 250.3 | 99.8 | 1.25 | 28.1 | 6.9 | 3.00 | 272.8 | 93.0 | 1.25 |
| 10 | 332.1 | 129.0 | 0.75 | 93.6 | 17.7 | 5.00 | 258.3 | 62.9 | 1.25 |
| 11 | 269.5 | 117.0 | 1.25 | 14.5 | 4.0 | 3.00 | 158.7 | 49.4 | 1.00 |
| 12 | 207.3 | 64.0 | 1.00 | 152.9 | 40.9 | 4.00 | 237.8 | 53.2 | 1.00 |
| Mean | 261.6 | 91.0 | 1.33 | 74.4 | 18.5 | 3.08 | 239.3 | 71.5 | 1.35 |
| Stddev | 95.2 | 32.5 | 0.43 | 45.2 | 12.1 | 1.14 | 40.1 | 15.8 | 0.32 |
| Max | 486.6 | 149.0 | 2.5 | 152.9 | 40.9 | 5.00 | 301.3 | 93.3 | 1.75 |
| Min | 133.0 | 37.6 | 0.75 | 14.5 | 4.0 | 2.00 | 158.7 | 49.4 | 1.00 |
| N | 12 | 12 | 12 | 12 | 12 | 12 | 10 | 10 | 10 |

Table 6 summarizes the pharmacokinetic parameters for each subject for each treatment with the arithmetic mean and standard deviation. A comparison of Treatment C with Treatment A showed similar mean values for AUC₀₋ₜ 239.3 (ng*hr/ml) vs. 261.6 (91%) and a lower value for Cₘₐₓ 71.5 (ng/ml) vs. 91.0 (79%). The results demonstrated a better PK profile for Treatment C. As with the log-transformed data, the variability of Treatment C was improved over that of Treatment A, wherein the standard deviation of the AUC was 40.1 vs. 95.2 and the standard deviation of the Cₘₐₓ values was 15.8 vs. 32.5.

The analysis of minimum and maximum values for individual volunteers also demonstrated the superiority of the compositions of the invention. The % CV or standard deviation profiles demonstrated that Treatment C improved on a statistical basis for a population. In drug treatment each individual patient is important and variability that can cause non efficacy (low AUC) or high level of adverse events (high AUC or Cₘₐₓ), both of which should be avoided. The analysis of the individual values of AUC₀₋ₜ showed that the range of values obtained for the reference was 486 (ng*hr/ml) to 133, a spread of 353 units where the maximum value was 186% of the mean and the minimum value was 51% of the mean. In contrast, for Treatment C the range of values was 301 (ng*hr/ml) to 159, a spread of only 142 units (less than half) where the maximum value was 126% of the mean and the minimum value was 66% of the mean. For Cₘₐₓ, the range of the reference drug was 163 (ng/ml) to 41, a spread of 112 units, and the maximum value was 163% of the mean value. In contrast, for Treatment C the range was 93 (ng/ml) to 49, a spread of 54 units (less than half of the reference), and the maximum value was 130% of the mean value. The data indicated that even on an individual basis Treatment C was less likely to be found non effective (minimum AUC 66% of mean vs. 51% for Treatment A) because the minimum individual AUC was closer to the mean value, and less likely to cause adverse effects (maximum AUC 126% of mean vs. 186% for reference and maximum Cₘₐₓ 130% of mean vs. 163% for the reference) because the maximum individual AUC was closer to the mean values.

What is claimed is:

1. A composition for reducing the variability of the bioavailability of a drug comprising cyclosporine dissolved in an effective amount of menthol and at least one surface active agent.

2. The composition according to claim 1, wherein cyclosporine is present in an amount of about 5% to about 20% by weight.

3. The composition according to claim 1, wherein menthol is present in an amount of about 30% to about 80% by weight.

4. The composition according to claim 1, wherein the surface active agent is present in an amount of about 20% to about 60% by weight.

5. The composition according to claim 1, wherein the surface active agent comprises a first surface active agent and a second surface active agent.

6. The composition according to claim 5, wherein the first surface active agent is present in an amount of about 25% to about 35% by weight and the second surface active agent is present in an amount of about 10% to about 20% by weight.

7. A method for reducing the variability of the bioavailability of cyclosporine comprising dissolving cyclosporine in an effective amount of menthol in the presence of at least one surface active agent.

8. The method according to claim 7, cyclosporine is present in an amount of about 5% to about 20% by weight.

9. The method according to claim 7, menthol is present in an amount of about 30% to about 80% by weight.

10. The method according to claim 7, wherein the surface active agent comprises a first surface active agent and a second surface active agent.

11. The method according to claim 10, wherein the first surface active agent is present in an amount of about 25% to about 35% by weight and the second surface active agent is present in an amount of about 10% to about 20% by weight.

12. The method according to claim 7, further comprising administering the composition to a mammal.

13. The method according to claim 7, wherein the amount of menthol is sufficient to decrease the variability in the drug's bioavailability by about 10% or more of the relative standard deviation (CV %) of the area under the blood or plasma concentration versus time curve (AUC) when compared to a non-menthol containing formulation AUC.

14. A method for increasing the time that cyclosporine provides a therapeutically significant concentration in blood or plasma comprising dissolving cyclosporine in an effective amount of menthol in the presence of at least one surface active ingredient.

15. The method according to claim 14, wherein the amount of menthol is sufficient to extend the time that the drug provides a therapeutically significant concentration in blood or plasma by one hour or more.

16. A composition for improving the bioavailability of cyclosporine comprising about 10% by weight cyclosporine dissolved in about 40% menthol, about 34% Tween 80, and about 17% ducosate sodium, wherein the weights are in percents of the composition.

17. The composition according to claim 16, wherein the composition has an AUC_{I} of about 248 ng*hr/ml.

18. The composition according to claim 16, wherein the composition has a Cₘₐₓ of about 69.9 ng/ml.

19. A composition for improving the bioavailability of a drug comprising simvastatin dissolved in an effective amount of menthol.

20. The composition according to claim 19, wherein the simvastatin is present in an amount of about 10% by weight.

21. A composition for improving the bioavailability of a drug comprising simvastatin dissolved in an effective amount of menthol, wherein the composition has an average AUCₜ of about 181% as compared to a non-menthol containing formulation.

22. The composition according to claim 21, wherein the composition has an average AUC_{I} of about 200% as compared to a non-menthol containing formulation.

23. The composition according to claim 21, wherein the composition has an average Cₘₐₓ of about 143% as compared to a non-menthol containing formulation.

24. A composition for improving the bioavailability of a drug comprising simvastatin dissolved in an effective amount of menthol, wherein the composition has an average AUC_{I} of about 127% as compared to the sequential administration of simvastatin and menthol.

25. The composition according to claim 24, wherein the composition has an average Cₘₐₓ of about 138% as compared to the sequential administration of simvastatin and menthol.

26. A composition for improving the bioavailability of an active metabolite of a drug comprising simvastatin dissolved in an effective amount of menthol, wherein the composition has an average AUCₜ for simvastatin hydroxyacid of about 143% as compared to a non-menthol containing formulation.

27. The composition according to claim 26, where the composition has an average AUCₜ for the simvastatin hydroxyacid of about 124% as compared to the sequential administration of simvastatin and menthol.

28. The composition according to claim 26, where the composition has an average Cₘₐₓ for the simvastatin hydroxyacid of about 145% as compared to a non-menthol containing formulation.

29. The composition according to claim 26, where the composition has an average Cₘₐₓ for the simvastatin hydroxyacid of about 129% as compared to the sequential administration of simvastatin and menthol.

30. A composition for reducing the variability of the bioavailability of an active metabolite of a drug comprising simvastatin dissolved in an effective amount of menthol, where the composition has a % CV for AUCₜ for simvastatin hydroxyacid which is at least about 30% lower when compared to a non-menthol containing formulation.

31. A composition for improving the bioavailability of a drug comprising raloxifene HCl and an effective amount of menthol, wherein the raloxifene HCl and the menthol are administered concomitantly.

32. The composition according to claim 31, wherein the raloxifene is present in an amount of about 25% by weight of the composition.

33. A composition for improving the bioavailability of a drug comprising raloxifene HCl and an effective amount of menthol, wherein the raloxifene HCl and the menthol are administered concomitantly and the composition has an average AUC of about 108% as compared to a non-menthol containing formulation.

34. The composition according to claim 33, wherein the composition has an average Cₘₐₓ of about 136% as compared to a non-menthol containing formulation.
